(19) United States
(12) Patent Application Publication
(10) Pub. No.: US 2020/0029633 A1
(43) Pub. Date: Jan. 30, 2020

(54) FEMUR/HIP PROTECTION SYSTEM

(71) Applicant: Frank Bowles, Addison, TX (US)

(72) Inventor: Frank Bowles, Addison, TX (US)

(21) Appl. No.: 16/520,506

(22) Filed: Jul. 24, 2019

Related U.S. Application Data
(60) Provisional application No. 62/751,916, filed on Oct. 29, 2018.
(60) Provisional application No. 62/702,832, filed on Jul. 24, 2018.

(51) Int. Cl. A41D 13/05; A61F 5/01

(52) U.S. Cl. CPC A41D 13/0506; A41D 13/0525; A41D 13/015; A61F 5/0102; A41D 13/0556

(57) ABSTRACT

Embodiments of a femur and/or hip protection system are disclosed. The device comprises a waist belt; a posterior assembly coupled to waist belt and having a center pad; a first leg assembly coupled to the waist belt and positioned on one side of the posterior assembly having a first protective pad; a second leg coupled to the waist belt and positioned on the opposing side of the posterior assembly having assembly having a second protective pad; a first impact distributing hip component coupled to the first leg assembly; and a second impact distributing hip component coupled to the second leg assembly.

### CROSS REFERENCE TO RELATED APPLICATIONS

This application claims the benefit of the filing date of U.S. provisional patent application Ser. No. 62/702,832, entitled “Femur/Hip Protection System”, filed on Jul. 24, 2018, and U.S. provisional patent application Ser. No. 62/751,916, entitled “An Improved Femur/Hip Protection System”, filed on Oct. 29, 2018, of which all of the disclosures are hereby incorporated by reference for all purposes.

### TECHNICAL FIELD

The invention relates in general to garments worn for protection, and in particular to garments and devices for hip, knee, and sacrum area protection.

### BACKGROUND INFORMATION

For many older adults and their families and caregivers, the risk of falling and suffering a debilitating hip fracture is a source of growing concern. Hip fractures are one of the most devastating, costly and lifestyle changing events for aging adults. This is due to bones becoming thinner and weaker from calcium loss as a person ages often as a result of osteoporosis or loss of bone tissue. More than 1.5 million Americans have fractures annually because of osteoporosis. Women are more prone to osteoporosis than men and hip fractures are more common among women because of this fact.

Men are not immune and as they age, they suffer from falls as well. The occurrence of hip fractures increases with age, doubling for each decade after the age of 50. The majority of hip fractures happen to people older than the age of 60. Hip fractures account for approximately 300,000 United States hospital admissions of senior adults of the age of 65 and older. Statistics show the majority of hip fractures, fractures of the upper femur, fractures of the femoral neck or the intertrochanteric are usually caused by falls.

What is needed, therefore, is a device to reduce or eliminate impact fracture to the hips, patella (knee caps) and sacral area due to falls.

### SUMMARY

In response to these and other problems, in one embodiment, there is femur/hip protection system having a plurality of pads attached to a belt designed to be worn around the waist. Additionally, special rubber and impact distributing pads are positioned next to the hip area. The concave design and the firm but flexible material of the hip pads helps to distribute the impact of a fall to the surrounding areas of the hip and thigh muscles. Thus, the femur/hip protective system is designed to reduce the chances of a devastating hip fracture, as well as injury to the knee and tailbone.

The femur/hip protection system is designed to be worn on the outside of clothing. It is also designed to be comfortable so it can be worn at all times—even while in bed.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1 is a back elevation of a femur/hip protective system which incorporates aspects of the present invention as if laid out on a flat surface.

FIG. 2A is a front isometric view illustrating the embodiment in FIG. 1 where the femur/hip protective system is wrapped so that its ends joins one another as if the system is about to be worn by a user.

FIG. 2B is a back isometric view illustrating the femur/hip protective system of FIG. 2A.

FIG. 3A is a back elevation of a left leg protector component of the femur/hip protective system of FIG. 1.

FIG. 3B is a back elevation of a pad used in the left leg protector component of FIG. 3A.

FIG. 4A is a front elevation of a posterior protector component of the femur/hip protective system of FIG. 1.

FIG. 4B is a front elevation of a first or posterior pad used in the posterior protector component of FIG. 4A.

FIG. 4C is a front elevation of a second or sacrum pad used in the posterior protector component of FIG. 4A.

FIG. 5A is a front isometric view of an impact distributing hip protector component used in the leg protector components of FIG. 1.

FIG. 5B is a back isometric view of the impact distributing hip protector component of FIG. 5A.

FIG. 5C is a front elevation view of the impact distributing hip protector component of FIG. 5A.

FIG. 5D is a section view horizontally cut through the impact distributing hip protector component of FIG. 5A.

FIG. 5E is a side view of the impact distributing hip component of FIG. 5A.

FIG. 5F is a section view cut vertically through the impact distributing hip component of FIG. 5A.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

When directions, such as upper, lower, top, bottom, clockwise, counter-clockwise, are discussed in this disclosure, such directions are meant to only supply reference directions for the illustrated figures and for orientation of components in the figures. The directions should not be read to imply actual directions used in any resulting invention or actual use. Under no circumstances, should such directions be read to limit or impart any meaning into the claims unless such directions are actually used in the claims.

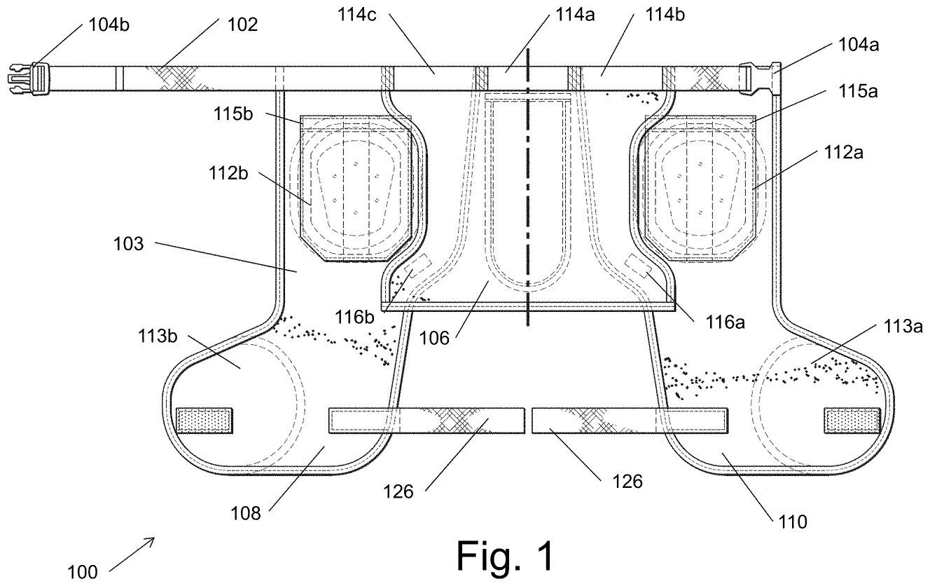

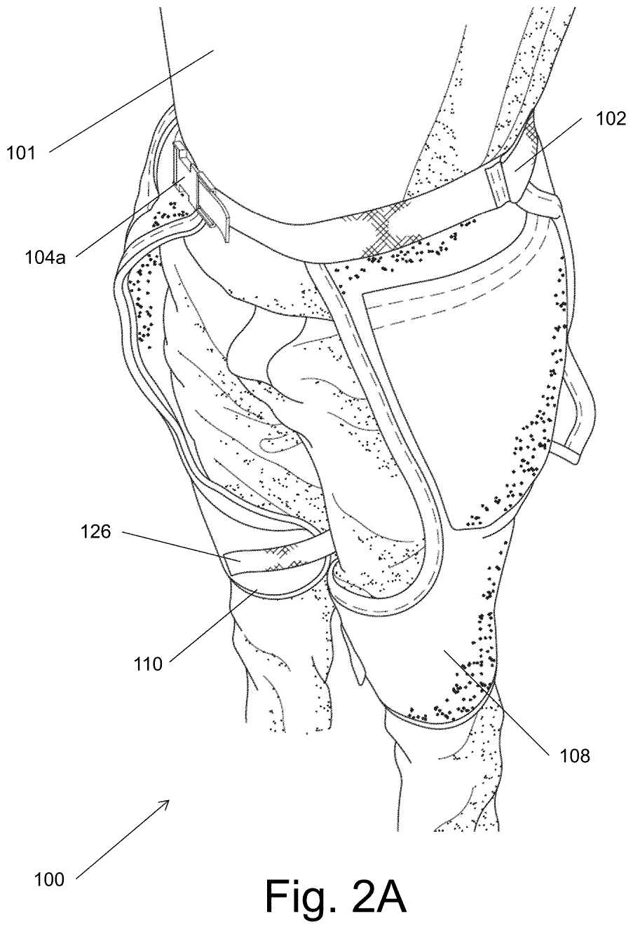

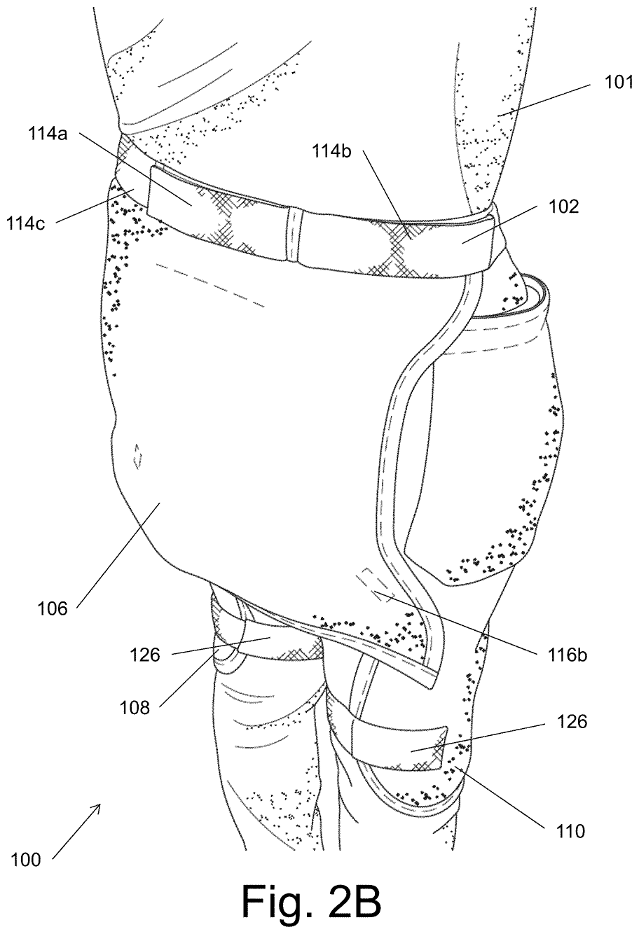

FIG. 1 illustrates one embodiment of a femur/hip protective system 100 which incorporates one or more aspects of the present invention. FIG. 1 is an illustration of a back elevation view as if the system is laid out on a flat surface. FIG. 2A is a front isometric view illustrating the femur/hip protective system 100 as may be worn by a user 101. FIG. 2B is a back isometric view illustrating the femur/hip protective system 100 as may be worn by the user 101.

Turning now to FIGS. 1, 2A and 2B, it can be seen that the femur/hip protective system 100 comprises a belt 102 designed to be worn around a user's waist. In certain embodiments, the belt 102 is adjustable and may be made from a strip of fabric webbing. In FIG. 1, the belt 102 extends from the left side of the main body or “apron” 103. In other embodiments (not shown), the belt 102 may extend from both sides of the apron 103. In certain embodiments, a female pinch clip 104a may be coupled to one end of the belt 102. A corresponding male pinch clip 104b may be coupled to the opposing end of the belt 102. Positioning the male pinch clip 104b longitudinally along the belt 102 allows for an overall adjustment of effective diameter of the belt 102 so that the belt can be adjusted for a wide variety of users. In other embodiments, both the female pinch clip 104a and the male pinch clip 104b may be adjusted longitudinally along the belt 102 to allow for overall adjustment of effective diameter of the belt.

In certain embodiments, there may be a posterior assembly 106 coupled to the belt 104 and positioned approximately in the center of the belt 102. A left leg assembly 108 may be coupled to the belt 102 and is positioned on one side of the center of belt 102. A right leg assembly 110 may also be coupled to the belt 102 and is positioned on the opposing side of the belt 104. As will be explained in detail below, the left leg assembly 108 and right leg assembly 110 may also be coupled to other protective pads, such as impact distributing hip components 112a and 112b (FIG. 1) and knee pads 113a and 113b (which are not generally visible from the back of the system or when a user is wearing the system).

In certain embodiments, there may be a rear handle 114a made from an additional piece of fabric webbing material. The rear handle 114a is designed to allow a single assistant accompanying the user to be able to quickly grab the user if the user loses his/her balance and begins to fall. In some embodiments, there may be additional rear handles 114b and 114c positioned on either side of the rear handle 114a. Rear handles 114b and 114c are designed to allow two assistants accompanying the user to be able to grab the belt 102 if the user begins to fall (or to be used to steady a user to prevent a fall incident altogether).

In certain embodiments, hook and loop material may be incorporated into various components of the femur/hip protective system 100. For instance, a piece of hook material 116a may be positioned on an inner surface of the posterior assembly 106 which aligns with a piece of loop material positioned on an outer surface of the right leg assembly 110 to couple a lower portion of the posterior assembly 106 to the leg assembly 110. Similarly, there may be another piece of hook material 116b positioned on an inner surface of the posterior assembly 106 which aligns with a piece of loop material positioned on an outer surface of the left leg assembly 108 to couple a lower portion of the posterior assembly 106 to the leg assembly 108. In alternative embodiments, the hook material 116a and 116b may be replaced with loop material and the loop material may be replaced with hook material and vice versa. It is generally irrelevant which connector is made from hook material and which connector is made from loop material. For purposes of this disclosure, a hook and loop connection system may employ both types of material. This disclosure uses all hook and loop material interchangeably.

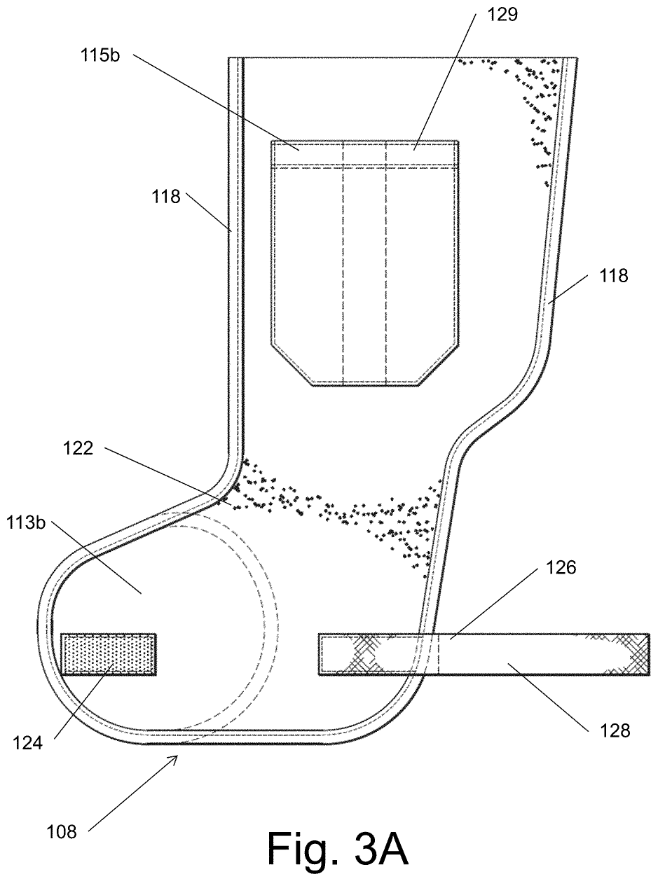

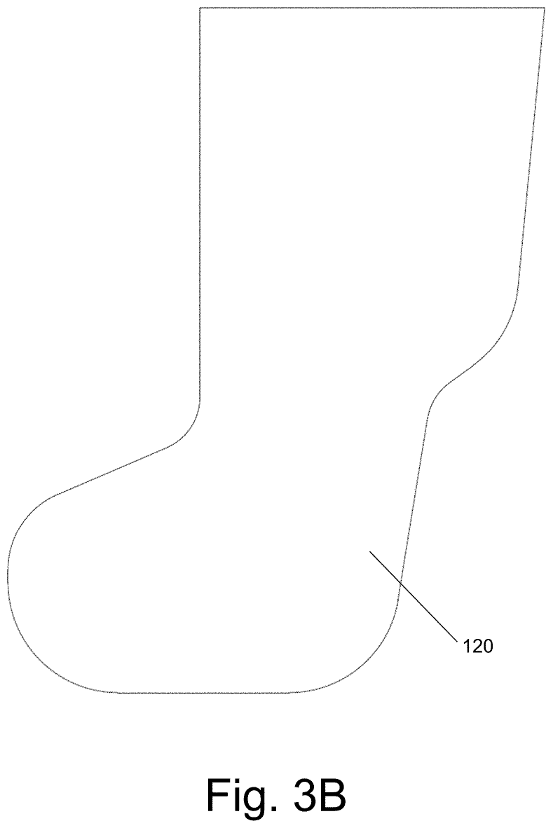

FIG. 3A is a back elevation of a left leg assembly 108 of the femur/hip protective system 100. In certain embodiments, the left leg assembly 108 comprises two pieces of fabric joined by a perimeter sewed edge 118 formed around a protective pad, such as the protective pad 120 illustrated in FIG. 3B. In certain embodiments, the protective pad may be a foam pad from ¼″ to ½″ in thickness. In alternative embodiments, the fabric could be a single piece being joined by a single seam or edge. In yet other embodiments, the fabric may be replaced by other material, such as plastic (or no material) and the edges formed by any known technique in the art, such as heat sealing.

As illustrated in FIGS. 3A and 3B, a lower portion 122 of the left leg assembly 108 widens in a lateral direction which tends to give the left leg assembly 108 an overall “boot like” shape in elevation. This widening of the lower portion 122 is designed to protect the user's knees from falls and any resulting injury. In certain embodiments, there may be an additional protective pad or extra layer of foam protection 113 positioned adjacent to the knee area to provide additional thickness for impact protection to the knees of the user. Such foam protection 113b may be coupled to an interior surface of the leg assemblies 108 and 110 and may be surrounded by fabric.

Loop material 124 (or in some embodiments, hook material) may be coupled to an exterior surface of the lower portion 122. On the opposing side of the lower portion 122, there may be a strap 126 formed of fabric material. On an interior surface of the strap 126, there may be sewed a piece of hook material 128 which, when folded around a user's knee (see FIGS. 2A and 2B) is designed to align with loop material 124. (In other embodiments where the exterior surface uses hook material, the strap may be coupled to loop material instead.) Thus, when the user folds the strap 126 over to align the hook material 128 with the loop material 124, the lower portion 122 is also wrapped around the user's knee—forming a protective pad around the user's knee (as illustrated in FIGS. 2A and 2B). The hook and loop connection system thus keeps the knee pad in place around the user's knee.

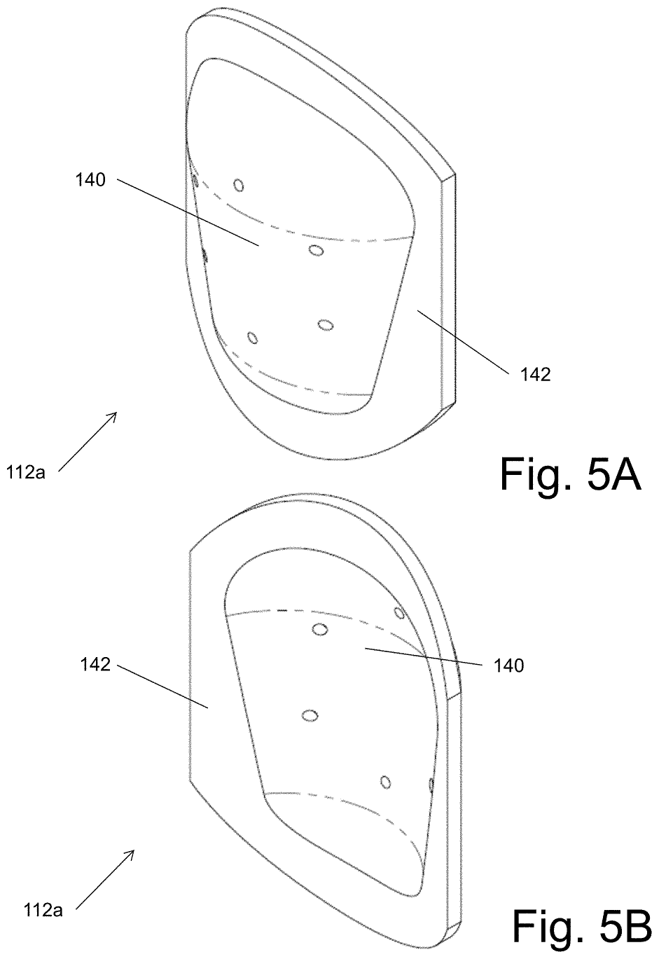

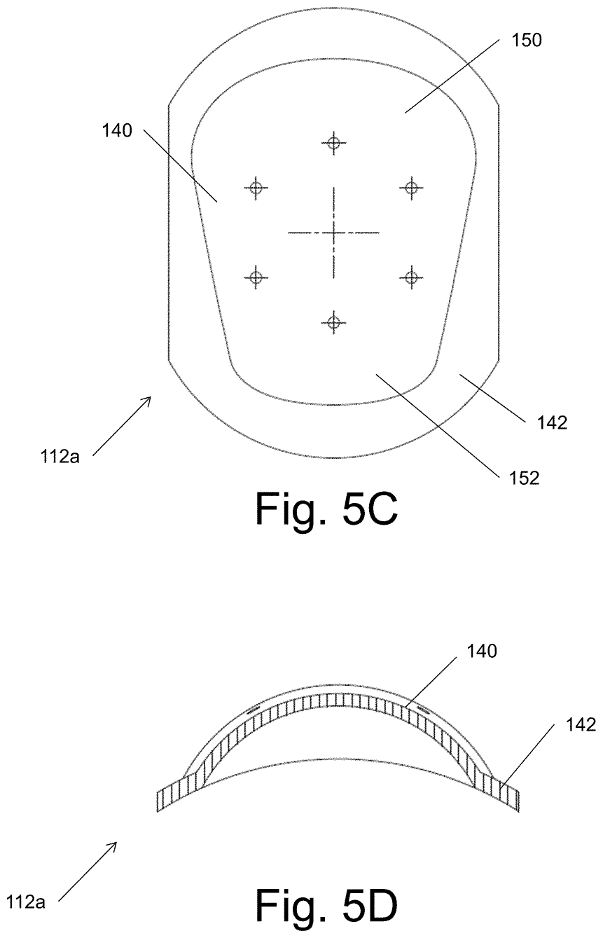

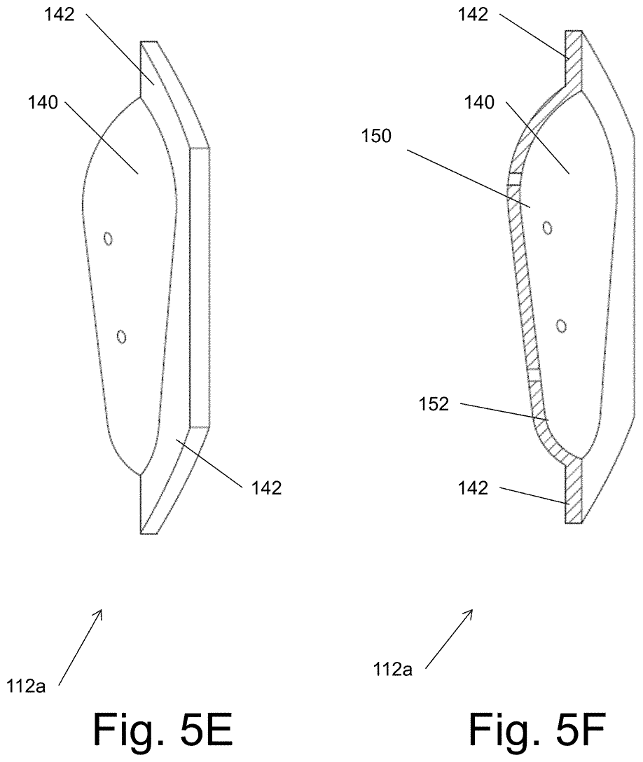

In certain embodiments, there may be a pocket 115b positioned on an exterior side of the left leg assembly 108 for holding the hip protector 112b. The details of the hip protector 112b will be discussed below in reference to FIGS. 5A through 5D.

The right leg assembly 110 generally mirrors the left leg assembly 108 and has similar details to the left leg assembly 108. The details of the right leg assembly 110 will not be discussed for purposes of brevity and clarity.

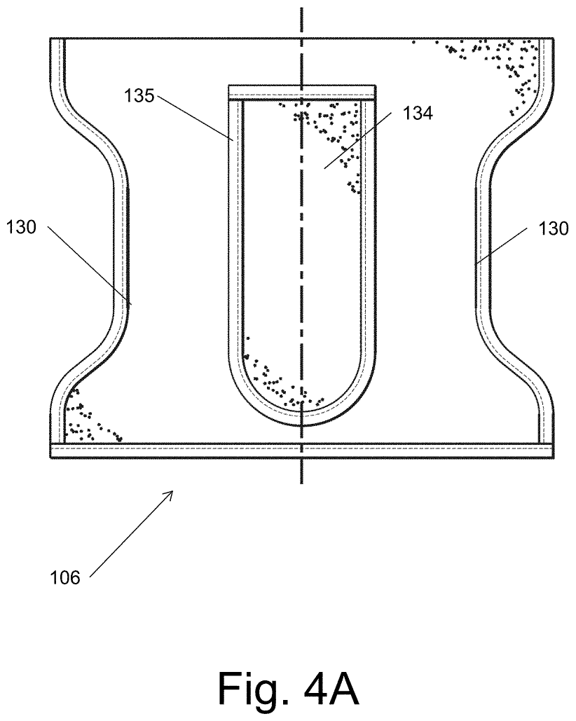

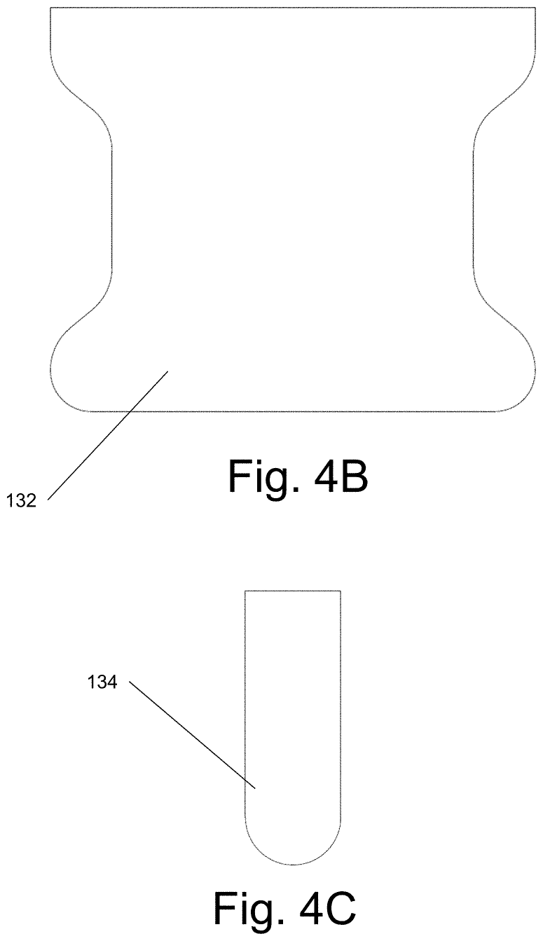

FIG. 4A is a front elevation of the posterior assembly 106 of the femur/hip protective system 100. In certain embodiments, the posterior assembly 106 comprises two pieces of fabric joined by a perimeter sewed edge 130 formed around a protective pad, such as the protective pad 132 illustrated in FIG. 4B. In certain embodiments, the protective pad 132 may be a foam pad from ¼″ to ½″ in thickness. In alternative embodiments, the fabric could be a single piece being joined by a single seam or edge. In yet other embodiments, the fabric may be replaced by other material, such as plastic (or no material) and the edges formed by any known technique in the art, such as heat sealing.

In certain embodiments, there may also be an additional protective pad 134 designed to offer protection to the sacrum area of the user. FIG. 4C is a front elevation view of the protective pad 134. The protective pad 134 is coupled to the interior surface of the posterior assembly 106 as illustrated in FIG. 4A and may be surrounded by fabric with a perimeter sewed edge 135. In certain embodiments, the protective pad 134 may be a foam pad from ¼″ to ½″ in thickness.

FIG. 5A is a front isometric view of the impact distributing hip component 112a (or impact distributing hip component 112b) which may be coupled to the left leg assembly 108 or the right leg assembly 110 via the pockets 115a and 115b. FIG. 5B is a back isometric view of the impact distributing hip component 112a. FIG. 5C is a front plan or elevation view of the impact distributing hip component 112a. FIG. 5D is a section view cut horizontally through the impact distributing hip component 112a. FIG. 5E is a side view of the impact distributing hip component 112a and FIG. 5F is a section view cut vertically through the impact distributing hip component 112a. Impact distributing hip components 112a and 112b are designed to fit within the exterior pockets 115a and 115b of the leg assemblies 110 and 108, respectively. In certain embodiments, a hook and loop system 129 may be employed on the top inner surfaces of the pockets 115a and 115b to secure the top of the pockets together which, thereby secures the position of the impact distributing hip components 112a and 112b.

Turning to FIGS. 5A to 5F, it can be seen that the impact distributing hip component 112a comprises a raised portion 140 protruding from a base portion 142. The raised portion 140 is designed to be aligned next to the femur area of a user. The raised portion 140 is positioned so that it faces away from the user. In other words, the base portion 142 is positioned within an exterior pocket 115a and 115b of the leg assemblies 108 and 110, respectively, such that the base portion 142 is adjacent to the user. If the user falls on the raised portion 140, the design of this embodiment allows for a distribution of pressure or force away from the femur area and distributes the impact to the surrounding areas of the hip and thigh muscles adjacent to the base portion 142. In certain embodiments, the impact distributing hip components 112a and 112b may be made from rubber or another impact resisting material with similar compressive and resilience properties.

In plan or elevation, such as illustrated in FIG. 5C, the raised portion 140 comprises a generally curved upper area 150 and a generally curved lower area 152. In a vertical direction, the curved lower area 152 is generally smaller than the upper area 150. When looking at a side view (FIG. 5E) and vertical section view (FIG. 5F), it can be seen that the curved upper area 150 protrudes a greater distance from the base portion 142 than that of the curved lower area 152. This shape allows for a more even distribution of force to areas away from a user's hip joint.

In yet, other embodiments, the impact distributing hip component is semi-spherical in shape. In other words, the protruding portion is part of a sphere coupled to a base portion (not shown).

### Operation

Referring now to FIGS. 1, 2A, 2B, and 3A, when a user wishes to use the femur/hip protective system 100, the user places the belt 102 around the user's waist. The user can then adjust the male pinch clip 104b and/or the female pinch clip 104a so that the belt 102 fits snuggly, but comfortably, around the user's waist.

The user may then wrap the strap 126 (FIG. 3A) around the user's knee so that the hook material 128 is aligned with the loop material 124. Pressing the hook material into the loop material 124 will secure the strap so that the lower portion 122 of the leg assembly 108 can provide protection for the knee. The user can repeat this process with the leg assembly 110 for the other knee. Once the belt 102 and the straps 126 are secured, the user may walk in a normal manner. If a user falls, the knee areas and hip areas are protected by padding as described above.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. When the word “means” is recited in a claim element, Applicant intends for the claim element to fall under 35 USC 112(f). Often a label of one or more words precedes the word “means.” The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC 112(f).

The foregoing description of the embodiments of the invention has been presented for the purposes of illustration and description. It is not intended to be exhaustive or to limit the invention to the precise form disclosed. Many combinations, modifications and variations are possible in light of the above teaching. For instance, in certain embodiments, each of the above described components and features may be individually or sequentially combined with other components or features and still be within the scope of the present invention. Undescribed embodiments which have interchanged components are still within the scope of the present invention. It is intended that the scope of the invention be limited not by this detailed description, but rather by the claims.

1. A femur/hip protective system comprising:
an adjustable waist belt;
a posterior assembly coupled to waist belt including
a center pad;
an additional pad coupled to an interior surface of the posterior assembly and sized and positioned to protect the sacral region of a user.
a first boot shaped leg assembly coupled to the waist belt and positioned on one side of the posterior assembly, the first boot shaped leg assembly including
a first protective pad;
a first strap having a connector for securing the first boot shaped leg assembly to the leg of a user;
a first additional knee pad coupled to an interior surface of the first leg assembly;
a second boot shaped leg assembly coupled to the waist belt and positioned on the opposing side of the posterior assembly, the second boot shaped leg assembly including
a second protective pad;
a second strap having a connector for securing the second boot shaped leg assembly to a second leg of a user;
a second additional knee pad coupled to an interior surface of the second leg assembly;
a first impact distributing hip component removeably positioned within a first pocket of the first leg assembly, wherein the first impact distributing hip component comprises a first curved raised portion protruding away from a first base portion and wherein the first curved raised portion has an upper portion and a lower portion such that the upper portion is wider than the lower portion and the upper portion protrudes farther from the base portion than the lower portion extends from the base portion; and
a second impact distributing hip component removeably positioned within a second pocket of the second leg assembly, wherein the second impact distributing hip component comprises a second curved raised portion protruding away from a second base portion.

2. A femur/hip protective system comprising:
a waist belt;
a posterior assembly coupled to waist belt and having a center pad;
a first leg assembly coupled to the waist belt and positioned on one side of the posterior assembly having a first protective pad;
a second leg assembly coupled to the waist belt and positioned on the opposing side of the posterior assembly having assembly having a second protective pad;
a first impact distributing hip component coupled to the first leg assembly; and
a second impact distributing hip component coupled to the second leg assembly.

3. The femur/hip protective system of claim 2, wherein the first impact distributing hip component and second impact distributing hip component comprise a curved raised portion protruding away from a base portion.

4. The femur/hip protective system of claim 3, wherein the curved raised portion has an upper portion and a lower portion such that the upper portion is wider than the lower portion.

5. The femur/hip protective system of claim 3, wherein the curved raised portion has an upper portion and a lower portion such that the upper portion protrudes farther away from the base portion than the lower portion protrudes from the base portion.

6. The femur/hip protective system of claim 2, wherein the first impact distributing hip component and second impact distributing hip component comprise a semi-spherical portion protruding away from a base portion.

7. The femur/hip protective system of claim 2, wherein the first leg assembly and the second leg assembly is shaped in the form of a boot to provide protection to the knee area of a user.

8. The femur/hip protective system of claim 2, wherein the first leg assembly or the second leg assembly has an additional knee pad coupled to an interior surface of the leg assembly to provide addition protection to the knee area.

9. The femur/hip protective system of claim 2, wherein the first leg assembly or the second leg assembly has a strap with a connector for wrapping around a portion of a user's leg.

10. The femur/hip protective system of claim 9, wherein the connector is part of a hook and loop connector.

11. The femur/hip protective system of claim 2, wherein the posterior assembly comprises an additional pad coupled to an interior surface of the posterior assembly and sized and positioned to protect the sacral region of a user.
